SEQUENCE LISTING (20 sequences)

SEQ ID NO: 1 - protein, 5 aa, Homo sapiens
KFERQ

SEQ ID NO: 2 - protein, 13 aa, Homo sapiens
GRKKRRQRRRPPQ

SEQ ID NO: 3 - protein, 15 aa, Homo sapiens
RQIKWFQNRRMKWKK

SEQ ID NO: 4 - protein, 21 aa, Homo sapiens
TRSSRAGLQFPVGRVHRLLRK

SEQ ID NO: 5 - protein, 13 aa, Homo sapiens
KRVSRNKSEKKRR

SEQ ID NO: 6 - protein, 18 aa, Homo sapiens
KLALKLALKALKAALKLA

SEQ ID NO: 7 - protein, 16 aa, Homo sapiens
AAVALLPAVLLALLAP

SEQ ID NO: 8 - protein, 20 aa, Homo sapiens
  MISC_FEATURE (1)..(20): Ku70-derived peptide
VPMLKEVPMLKPMLKEPMLK

SEQ ID NO: 9 - protein, 28 aa, Homo sapiens
MANLGYWLLALFVTMWTDVGLCKKRPKP

SEQ ID NO: 10 - protein, 18 aa, Homo sapiens
LLIILRRRIRKQAHAHSK

SEQ ID NO: 11 - protein, 21 aa, Homo sapiens
KETWWETWWTEWSQPKKKRKV

SEQ ID NO: 12 - protein, 18 aa, Homo sapiens
RGGRLSYSRRRFSTSTGR

SEQ ID NO: 13 - protein, 27 aa, Homo sapiens
GWTLNSAGYLLGKINLKALAALAKKIL

SEQ ID NO: 14 - protein, 21 aa, Homo sapiens
AGYLLGKINLKALAALAKKIL

SEQ ID NO: 15 - protein, 20 aa, Homo sapiens
GLWRALWRLLRSLWRLLWRA

SEQ ID NO: 16 - protein, 15 aa, Homo sapiens
SDLWEMMMVSLACQY

SEQ ID NO: 17 - protein, 12 aa, Homo sapiens
TSPLNIHNGQKL

SEQ ID NO: 18 - protein, 26 aa, Homo sapiens
DPKGDPKGVTVTVTVTVTGKGDPKPD

SEQ ID NO: 19 - protein, 16 aa, Homo sapiens
RVIRVWFQNKRCKDKK

SEQ ID NO: 20 - protein, 7 aa, Homo sapiens
RRRRRRR

(19) United States
(12) Patent Application Publication
(10) Pub. No.: US 2013/0065267 A1
(43) Pub. Date: Mar. 14, 2013

(54) CONJUGATES AND METHODS FOR MEASURING CHAPERONE-MEDIATED AUTOPHAGY

(71) Applicant: Zixu Mao, Atlanta, GA (US)

(72) Inventor: Zixu Mao, Atlanta, GA (US)

(73) Assignee: EMORY UNIVERSITY, Atlanta, GA (US)

(21) Appl. No.: 13/602,558

(22) Filed: Sep. 4, 2012

Related U.S. Application Data
(60) Provisional application No. 61/532,688, filed on Sep. 9, 2011.

(51) Int. Cl. G01N 21/77; C07K 19/00

(52) U.S. Cl. 435/29; 530/350

(57) ABSTRACT

This disclosure relates to methods of detecting chaperone-mediated autophagy. In some embodiments, the disclosure relates to methods of measuring chaperone-mediated autophagy in living cells and in purified lysosomes. In some embodiments, the disclosure relates to methods of detecting, diagnosing, monitoring, and/or treating lysosomal diseases in a subject.

### CROSS REFERENCE TO RELATED APPLICATIONS

This application claims priority to U.S. provisional application No. 61/532,688 filed Sep. 9, 2011, hereby incorporated by reference in its entirety.

### ACKNOWLEDGEMENTS

This invention was made with government support under Grants AG023695 and NSO48254 awarded by the National Institutes of Health. The government may have certain rights in the invention.

### FIELD

This disclosure relates to methods of detecting chaperone-mediated autophagy. In some embodiments, the disclosure relates to methods of measuring chaperone-mediated autophagy in living cells and in purified lysosomes. In some embodiments, the disclosure relates to methods of detecting, diagnosing, monitoring, or treating lysosomal diseases in a subject.

### BACKGROUND

Lysosomes are organelles present in animal cells that play an important role in maintaining cellular homeostasis. They accomplish this by controlling the turnover of various intracellular components, including cell debris, engulfed viruses, engulfed bacteria, misfolded proteins, and other matter, by acid hydrolases. This process is termed autophagy. Autophagy is believed to be crucial during periods of starvation for cells to recycle nutrients with which to survive and may be upregulated during conditions of stress. There are three major types of autophagy, namely macroautophagy, microautophagy, and chaperone-mediated autophagy (CMA).

Macroautophagy is a process by which a cell processes long-lived proteins or damaged organelles. The target to be degraded is enveloped by an autophagosome, a vesicle with a double membrane. The autophagosome's outer membrane joins with a lysosome to form an autophagolysosome wherein the contents are degraded by acid hydrolases. Microautophagy is a process by which the lysosomal limiting membrane directly envelops cytosolic material to be degraded. This cytosolic material may include organelles, such as the nucleus or peroxisomes.

In contrast to the relatively non-selective nature of the two other forms of autophagy, chaperone-mediated autophagy (CMA) is distinguished by its high selectivity, degrading particular cytosolic proteins via a specific recognition motif, KFERQ, on its substrates. CMA is largely controlled by a group of chaperone proteins including chaperone heat shock cognate 70 (Hsc70) and lysosome-associated membrane protein 2A (Lamp2a). Hsc70 recognizes the KFERQ motif on a protein and transports it to the lysosomal membrane, where Lamp2a translocates the protein into the lysosome for degradation. CMA activity is closely regulated by a number of physiological and pathological signals.

Dysregulation of autophagy has been shown to underlie the pathogenic processes of many human diseases. Dysregulation of CMA in particular may occur in cancer, immunologic disorders, infectious diseases, neurodegenerative diseases, and aging. Modulation of CMA or targeting of its substrate has been shown to restore tissue function in aging and to reduce neurotoxicity in models of Huntington's Disease.

Currently, measuring CMA activity typically requires the technically challenging, complicated, and time-consuming isolation of lysosomes from cells or tissues. See, e.g., Kaushik S, Cuervo A M., Chaperone-mediated autophagy. Methods Mol Biol. 2008;445:227-44. Methods not requiring lysosomal isolation are time-consuming, require complicated expression of modified proteins within the target cell, require multiple components, and/or do not permit dynamic monitoring of cells, making the techniques impractical for clinical diagnostic purposes. See, e.g., US Published Patent Application Nos. US 2005/0277116 A1, US 2006/0014712, and US 2003/0190684 A1. In addition, methods of measuring CMA activity are often low-throughput and cannot be used to measure CMA activity in live, unmodified cells. There is a need for a method of detecting CMA activity that is quick, sensitive, easy-to-use, and capable of dynamically monitoring live cells.

### SUMMARY

This disclosure relates to methods of detecting chaperone-mediated autophagy. In some embodiments, the disclosure relates to methods of measuring chaperone-mediated autophagy in living cells and in purified lysosomes. In some embodiments, the disclosure relates to methods of detecting, diagnosing, monitoring, or treating lysosomal diseases in a subject.

In some embodiments, the disclosure relates to a conjugate, comprising a fusion of the KFERQ peptide, a membrane-permeable peptide, and a marker. In some embodiments, the KFERQ peptide is replaced with a peptide of substantial similarity. In some embodiments, the membrane-permeable peptide may include, but is not limited to, a peptide with an amino acid sequence listed in SEQ ID NO: 2, 3, 4, 5, 6, 7, 8, 9, 10, 11, 12, 13, 14, 15, 16, 17, 18, 19, or 20 or a peptide with substantial similarity to any of those sequences. In some embodiments, the marker is a fluorescent dye. In some embodiments, the marker is a polycyclic aromatic dye, metal complex, nanoparticle, or fluorescent protein. In some embodiments, the marker is a fluorescein or rhodamine dye. In some embodiments, the fluorescent protein may include, but is not limited to, green fluorescent protein (GFP), cyan fluorescent protein (CFP), red fluorescent protein (RFP), or yellow fluorescent protein (YFP). In some embodiments, the marker comprises more than one fluorescent proteins.

In some embodiments, the disclosure relates to a method for direct and specific measurement of CMA activity. In some embodiments, the method measures CMA activity in vitro. In some embodiments, the method measures CMA activity in vivo. In some embodiments, the disclosure is a method of detecting the measurement of CMA activity in cells by correlating the CMA activity with the level, signal, or intensity of the conjugate marker signal(s) within the cells. In some embodiments, the disclosure relates to a method of detecting CMA in a cell, comprising mixing one or more cells and a conjugate comprising a peptide comprising KFERQ (SEQ ID NO: 1) or a peptide with substantial similarity to KFERQ, a membrane-permeable peptide, and a marker and analyzing the cell(s) for the marker.

In some embodiments, the disclosure is a method of analyzing one or more cells comprising exposing the cell to light and detecting light coming from the cell after mixing the cell(s) with a conjugate disclosed herein. In some embodiments, the disclosure relates to a method wherein detecting a signal from the marker indicates CMA activity has not occurred. In some embodiments, the disclosure is a method wherein detecting the absence of a signal indicates that CMA activity has occurred.

In some embodiments, the disclosure is a method of high throughput and/or high content screening for drug discovery, applied to tissues and/or cells for a diagnostic purpose. In some embodiments, the disclosure relates to a method of detecting the inhibition of CMA activity in a cell, comprising mixing a cell with a test compound, then mixing the cell and a conjugate comprising a peptide comprising KFERQ (SEQ ID NO: 1) or peptide of substantial similarity, a membrane-permeable peptide, and a marker, then analyzing the cell for the marker, and correlating a change in the marker with the test compound, with a decrease in marker being indicative of CMA-inhibition mediated by the test compound.

In some embodiments, the disclosure is a method for diagnosing, monitoring, detecting, and/or treating lysosomal diseases in a subject including, but not limited to, Aspartylglucosaminuria, Fucosidosis, a-Mannosidosis, b-Mannosidosis, Mucolipidosis I (sialidosis), Schindler disease, Fabry's disease, Farber's disease, Gaucher's disease, GM1 gangliosidosis, Tay-Sachs disease, Sandhoff's disease, Krabbe's disease, Metachromatic leukodystrophy, Niemann-Pick disease, types A and B, Niemann-Pick disease type C, Wolman's disease, Neuronal ceroid lipofuscinosis, Glycogen storage disease, Glycogen storage disease type II (Pompe's disease), Multiple enzyme deficiency, Multiple sulphatase deficiency, Galactosialidosis, Mucolipidosis II/III, Mucolipidosis IV, Lysosomal transport defects, Cystinosis, Sialic acid storage disease, Danon disease, Hyaluronidase deficiency, the Mucopolysaccharidoses (MPS) MPS I, MPS II, MPS IIIA, MPS IIIB, MPS IIIC, MPS IIID, MPS IVA, MPS IV B, MPS VI, and MPS VII, and any other lipidoses, Glycoproteinoses, Sphingolipidoses, or other disorders due to defects in lysosomal proteins.

### DETAILED DESCRIPTION

### Chaperone-Mediated Autophagy (CMA)

As used herein, the term CMA may refer to either a portion or the entire process of chaperone-mediated autophagy. CMA is characterized by the preferential degradation of substrates containing a particular recognition motif, as described herein. The amino acid sequence of the CMA recognition motif is set forth in SEQ ID NO: 1, KFERQ. The recognition motif may also be a variant with substantial similarity to KFERQ, as described herein.

### Cell-Penetrating Peptides

The amino acid sequence of the cell-penetrating peptide derived from the human immunodeficiency virus (HIV)-1 Tat protein residue 48-60 is set forth in SEQ ID NO: 2, GRKKRRQRRRPPQ.

The amino acid sequence of Antp (Drosophila Antennapedia-(43-58)), also known as Penatratin, is set forth in SEQ ID NO: 3, RQIKWFQNRRMKWKK.

The amino acid sequence of Buforin II, a peptide capable of translocating across liposome membranes, is set forth in SEQ ID NO: 4, TRSSRAGLQFPVGRVHRLLRK.

The amino acid sequence of hClock-(35-47) (human Clock protein DNA-binding peptide), which has translocation activity similar to (HIV)-1 Tat protein residue 48-60, is set forth in SEQ ID NO: 5, KRVSRNKSEKKRR.

The amino acid sequence of MAP (cell-penetrating, model amphipathic peptide) is set forth in SEQ ID NO: 6, KLALKLALKALKAALKLA.

The amino acid sequence of Kaposi's sarcoma fibroblast growth factor (K-FGF) is set forth in SEQ ID NO: 7, AAVALLPAVLLALLAP.

The amino acid sequence of a cell-permeable peptide derived from the Bax-binding domain of the DNA repair factor Ku70 is set forth in SEQ ID NO: 8, comprising a peptide selected from the group comprising VPMLKE, VPMLK, PMLKE and PMLK.

The amino acid sequence of prion, Mouse PrP^{c} (1-28), is set forth in SEQ ID NO: 9, MANLGYWLLALFVTMWTDVGLCKKRPKP.

The amino acid sequence of pVEC, a cell-penetrating peptide derived from the murine vascular endothelial-cadherin protein, is set forth in SEQ ID NO: 10, LLIILRRRIRKQAHAHSK.

The amino acid sequence of Pep-1, a cell-penetrating peptide, is set forth in SEQ ID NO: 11, KETWWETWWTEWSQPKKKRKV.

The amino acid sequence of SynB1 is set forth in SEQ ID NO: 12, RGGRLSYSRRRFSTSTGR.

The amino acid sequence of Transportan is set forth in SEQ ID NO: 13, GWTLNSAGYLLGKINLKALAALAKKIL.

The amino acid sequence of Transportan-10 is set forth in SEQ ID NO: 14, AGYLLGKINLKALAALAKKIL.

The amino acid sequence of CADY is set forth in SEQ ID NO: 15, Ac-GLWRALWRLLRSLWRLLWRA-cysteamide.

The amino acid sequence of Pep-7 is set forth in SEQ ID NO: 16, SDLWEMMMVSLACQY.

The amino acid sequence of HN-1 is set forth in SEQ ID NO: 17, TSPLNIHNGQKL.

The amino acid sequence of VT5 is set forth in SEQ ID NO: 18, DPKGDPKGVTVTVTVTVTGKGDPKPD.

The amino acid sequence of pISL is set forth in SEQ ID NO: 19, RVIRVWFQNKRCKDKK.

The amino acid sequence of (R)₇ is set forth in SEQ ID NO: 20, RRRRRRR^{C}.

As used herein, “subject” refers to any animal, preferably a human patient, livestock, or domestic pet.

As used herein, an unspecified “R” group is an unspecified amino acid. As used herein, (R)_{N} is a sequence of “N” number of R repeats, where R is a particular, unspecified amino acid. For example (K)₅ is a peptide comprising Lysine-Lysine-Lysine-Lysine-Lysine.

As applied to polypeptides, the term “substantial similarity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “protein” and “peptide” refer to compounds comprising amino acids joined via peptide bonds and are used interchangeably. As used herein, “amino acid sequence” may refer to an amino acid sequence of a protein molecule. However, terms such as “peptide” or “protein” may include post-translational modifications of the amino acid sequences, such as amino acid deletions, additions, and modifications such as glycosylations and addition of lipid moieties.

The term “fusion” when used in reference to a protein or peptide refers to a chimeric protein containing a protein of interest joined to one or more peptides (the fusion partner). The fusion partner may serve various functions, including enhancement of solubility or membrane-penetration of the peptide of interest, as well as providing an “affinity tag” to allow purification of the recombinant fusion peptide from a host cell or from a supernatant or from both. The fusion partner may comprise a marker. If desired, the fusion partner may be removed from the protein of interest after or during purification.

### EXPERIMENTAL

### Example 1

### Conjugate for the Detection of CMA Activity

CMA activity was measured by means of administering a tripartite fusion protein comprising a KFERQ recognition motif, a (HIV)-1 Tat protein residue 48-60 as set forth in SEQ ID NO: 2, and green fluorescent protein (GFP) to an animal, typically mammalian, cell culture. The (HIV)-1 Tat protein residue enabled the fusion protein to cross the cell membrane and enter into the cytosol. The KFERQ motif was recognized by Hsc70. The Hsc70 in turn transported the fusion protein to the lysosomal membrane, where Lamp2a translocated the protein into the lysosome. After a period of incubation of the fusion protein with the cell culture, the cells were analyzed by confocal microscopy for localization of GFP expression to the lysosomes.

### Example 2

### Method of Detecting Lysosomal Disease by Measuring CMA Activity

Huntington's disease has been associated with defects in autophagy, including CMA. Harnessing CMA has been shown to ameliorate aspects of this disease. A method for diagnosing CMA defects in patients either diagnosed or suspected of being afflicted with Huntington's Disease will comprise obtaining a cell sample from the patient, culturing the cells, incubating the cells with a fusion protein comprising the KFERQ motif, the cell-penetrating peptide Pep-1 (SEQ ID NO: 11), and yellow fluorescent protein (YFP), and analyzing the intensity of YFP localization to the lysosomes within the cells. This method may be performed serially as a means of detecting disease progression, remission, or maintenance.

### Example 3

### High-throughput Screening of Potential CMA Inhibitors

A method of screening for CMA inhibitors will comprise culturing animal, typically mammalian, cells with a candidate CMA inhibitor at a range of concentrations (including 0 mg/mL, referred to as a blank) either prior to or concurrent with addition of a fusion protein comprising a KFERQ motif, MAP (SEQ ID NO: 6), and red fluorescent protein (RFP). After a period of incubation, the intensity of RFP localization to the lysosome will be analyzed by confocal microscopy and will be analyzed in light of the varying concentrations of the candidate inhibitor that will be used. CMA inhibition will be indicated by a reduction in RFP signal intensity in the presence of the candidate inhibitor compared to that seen in the blank sample.

What is claimed:

1. A conjugate, comprising:
a. a peptide, comprising KFERQ (SEQ ID NO: 1) or a peptide with substantial similarity to KFERQ;
b. a membrane-permeable peptide; and
c. a marker.

2. The conjugate of claim 1, wherein the membrane-permeable peptide is selected from the group, comprising:
a. TAT (transactivator of transcription), human immunodeficiency virus type-1 (HIV-1) Tat-(48-60), GRKKRRQRRRPPQ (SEQ ID NO: 2);
b. Antp (Drosophila Antennapedia-(43-58)), RQIKWFQNRRMKWKK (SEQ ID NO: 3);
c. Buforin II, TRSSRAGLQFPVGRVHRLLRK (SEQ ID NO: 4);
d. hClock-(35-47) (human Clock protein DNA-binding peptide), KRVSRNKSEKKRR (SEQ ID NO: 5);
e. MAP (model amphipathic peptide), KLALKLALKALKAALKLA (SEQ ID NO: 6);
f. K-FGF, AAVALLPAVLLALLAP (SEQ ID NO: 7);
g. Ku70-derived peptide, comprising a peptide selected from the group comprising VPMLKE, VPMLK, PMLKE and PMLK (SEQ ID NO: 8);
h. Prion, Mouse PrP^{c} (1-28), MANLGYWLLALFVTMWTDVGLCKKRPKP (SEQ ID NO: 9);
i. pVEC, LLIILRRRIRKQAHAHSK (SEQ ID NO: 10);
j. Pep-1, KETWWETWWTEWSQPKKKRKV (SEQ ID NO: 11);
k. SynBl, RGGRLSYSRRRFSTSTGR (SEQ ID NO: 12);
l. Transportan, GWTLNSAGYLLGKINLKALAALAKKIL (SEQ ID NO: 13);
m. Transportan-10, AGYLLGKINLKALAALAKKIL (SEQ ID NO: 14);
n. CADY, Ac-GLWRALWRLLRSLWRLLWRA-cysteamide (SEQ ID NO 15);
o. Pep-7, SDLWEMMMVSLACQY (SEQ ID NO: 16);
p. HN-1, TSPLNIHNGQKL (SEQ ID NO: 17);
q. VT5, DPKGDPKGVTVTVTVTVTGKGDPKPD (SEQ ID NO: 18);
r. pISL, RVIRVWFQNKRCKDKK (SEQ ID NO: 19); and
s. (R)₇, RRRRRRR (SEQ ID NO: 20).

3. The conjugate of claim 1, wherein the marker is a polycyclic aromatic dye, metal complex, nanoparticle, or a protein.

4. The conjugate of claim 1, wherein the marker is fluorescent.

5. The conjugate of claim 1, wherein the marker is a fluorescent protein or rhodamine dye.

6. The conjugate of claim 5, wherein the fluorescent protein is green fluorescent protein (GFP), cyan fluorescent protein (CFP), red fluorescent protein (RFP), or yellow fluorescent protein (YFP).

7. The conjugate of claim 5 further comprising a second fluorescent protein.

8. The conjugate of claim 7 wherein the first dye is a cyan fluorescent protein (CFP) and the second dye is yellow fluorescent protein (YFP).

9. A method of detecting chaperone-mediated autophagy in a cell, comprising:
a. mixing a cell and a conjugate comprising a peptide comprising KFERQ (SEQ ID NO: 1) or a peptide with substantial similarity to KFERQ, a membrane-permeable peptide, and a marker; and
b. analyzing the cell for the marker.

10. The method of claim 9, wherein the marker is a fluorescent dye.

11. The method of claim 9, wherein analyzing the cell for the marker comprises exposing the cell to light and detecting light coming from the cell.

12. The method of claim 9, wherein detecting a signal from the marker indicates chaperone-mediated autophagy has not occurred.

13. The method of claim 9, wherein detecting the absence of a signal indicates that chaperone-mediated autophagy has occurred.

14. The method of claim 9, wherein the subject is at risk for, is suspected of having, has been diagnosed with, is being treated for, or is in remission from lysosomal disease.

15. A method of detecting the inhibition of chaperone-mediated autophagy in a cell, comprising:
a. mixing a cell with a test compound;
b. mixing the cell and a conjugate comprising a peptide comprising KFERQ (SEQ ID NO: 1), a membrane-permeable peptide, and a marker;
c. analyzing the cell for the marker; and
d. correlating a change in the marker with the test compound, with a decrease in marker being indicative of CMA inhibition mediated by the test compound.
